SEQUENCE LISTING (11 sequences)

SEQ ID NO: 1 - DNA, 100 bp, Artificial Sequence
  taget sequence of MLH1 - genomic sequence
gtcgagccgggctcacttaagggctacgacttaacgggccgcgtcactcaatggcgcggacacgcctctttgcccgggcagaggcatgtacagcgcatgc

SEQ ID NO: 2 - DNA, 100 bp, Artificial Sequence
  target sequence of MLH1 after bisulphite treatment.
gtcgagtcgggtttatttaagggttacgatttaacgggtcgcgttatttaatggcgcggatacgtttttttgttcgggtagaggtatgtatagcgtatgt

SEQ ID NO: 3 - DNA, 26 bp, Artificial Sequence
  primer MLH1MSHRMF1
gtcgagtcgggtttatttaagggtta

SEQ ID NO: 4 - DNA, 25 bp, Artificial Sequence
  MLH1MSHRMR1
acatacgctatacatacctctaccc

SEQ ID NO: 5 - DNA, 100 bp, Artificial Sequence; identical to SEQ ID NO: 2
  MLH1_METHYLATED_CONTROL

SEQ ID NO: 6 - DNA, 100 bp, Artificial Sequence
  MLH1_NON_METHYLATED_CONTROL
gttgagttgggtttatttaagggttatgatttaatgggttgtgttatttaatggtgtggatatgtttttttgtttgggtagaggtatgtatagtgtatgt

SEQ ID NO: 7 - DNA, 2752 bp, Artificial Sequence
  pUCIDT-Amp (plasmid)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgcggccgccggagggttgcgtttgagacgggcgacagatatcagttctggaccagcgagctgtgctgcgacgcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctctttcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 2056 bp, Artificial Sequence
  pIDTSmart-Amp (plasmid)
cccgtgtaaaacgacggccagtttatctagtcagcttgattctagctgatcgtggaccggaaggtgagccagtgagttgattgcagtccagttacgctggagtctgaggctcgtcctgaatgatatgcggccgccggagggttgcgtttgagacgggcgacagatatcagttctggacgagcgagctgtcgtccgacgcgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 9 - DNA, 2705 bp, Artificial Sequence
  pUCIDT-Kan (plasmid)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaagtgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaagagtttatgcatttctttccagacttgttcaacaggccagccattacgctcgtcatcaaaatcactcgcaccaaccaaaccgttattcattcgtgattgcgcctgagcgagacgaaatacgcgatcgccgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagcctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcaagcaagacgtttcccgttgaatatggctcattttagcttccttagctcctgaaaatctcgataactcaaaaaatacgcccggtagtgatcttatttcattatggtgaaagttggaacctcttacgtgccgatcacacatttccccgaaaagtgccacctgacgtctaagaaaccattattatcatgacattaacctataaaaataggcgtatcacgaggccctttcgtc

SEQ ID NO: 10 - DNA, 1962 bp, Artificial Sequence
  pIDTSmart-Kan (plasmid)
cccgtgtaaaacgacggccagtttatctagtcagcttgattctagctgatcgtggaccggaaggtgagccagtgagttgattgcagtccagttacgctggagtctgaggctcgtcctgaatgatatgcggccgccggagggttgcgtttgagacgggcgacagatatcactactggaccgcgagctgtgctgcgacgcgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 11 - DNA, 3170 bp, Artificial Sequence
  pBRIDT (plasmid)
cgaggccctttcgtcttcaagcattctcatgtttgacagcttatcaccgatatcctttaatgcggtagtttatcacagttaaattgctaacgcagtcaggcaccgtgtatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatatcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccttatgcggtgtgaaataccgcacagatgcgtaaggagaaaataccgcatcaggcgctcttacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgaacgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttactttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatca

(19) United States
(12) Patent Application Publication
(10) Pub. No.: US 2022/0010375 A1
(43) Pub. Date: Jan. 13, 2022

(54) CONTROL PLASMIDS AND USES THEREOF

(71) Applicant: Aarhus Universitet, Aarhus C (DK)

(72) Inventors: Lise Lotte Hansen, Risskov (DK); Tomasz K. Wojdacz, Nyborg (DK)

(21) Appl. No.: 17/413,145

(22) PCT Filed: Dec. 13, 2019
(86) PCT No.: PCT/EP2019/085106

§ 371 Date: Jun. 11, 2021

(30) Foreign Application Priority Data
Dec. 14, 2018 (EP) 18212541.9

(51) Int. Cl. C12Q 1/6876; C12N 15/85

(52) U.S. Cl. CPC C12Q 1/6876; C12Q 2600/154; C12N 15/85

(57) ABSTRACT

The present invention relates to a set of references nucleic acids for use in a method of detecting methylated CpG-containing nucleic acids by nucleic acid amplification and preferably melting curve analysis of amplification products.

### FIELD OF THE INVENTION

The present invention relates to a set of references nucleic acids for use in a method of detecting methylated CpG-containing nucleic acids by nucleic acid amplification and preferably melting curve analysis of amplification products.

### BACKGROUND OF THE INVENTION

DNA methylation is a heritable, reversible and epigenetic change of the DNA sequence without altering its coding function. DNA methylation harbours the potential to alter gene expression which in turn affects developmental and genetic processes. The methylation reaction involves flipping a target cytosine out of an intact double helix thereby allowing the transfer of a methyl group from S-adenosylmethionine in a cleft of the enzyme DNA (cytosine-5)-methyltransferase to form 5-methylcyto sine (5-mCyt). This enzymatic conversion is the only epigenetic modification of DNA known to exist in vertebrates and is essential for normal embryonic development.

CpG islands (CpG-rich sequences) are distributed across the human genome and often span the promoter region as well as the first exon of protein coding genes. Methylation of individual promoter region CpG islands usually turns off or reduce the rate of transcription by recruiting histone deacetylases, which supports the formation of inactive chromatin. CpG islands are typically between 0.2 to about 1 kb in length and are located upstream of many housekeeping and tissue-specific genes, but may also extend into gene coding regions. Therefore, it is the methylation of cytosine residues within CpG islands in somatic tissues, which is believed to affect gene function by altering transcription.

Abnormal methylation of CpG islands associated with tumor suppressor genes may also cause decreased gene expression. Increased methylation of such regions may lead to progressive reduction of normal gene expression giving abnormal cells growth (i.e., a malignancy).

Methylation promoter regions, particularly in tumour suppressor genes, and genes involved in apoptosis and DNA repair, is one of the hallmarks of cancer. Changes in the methylation status of these genes are an early event in cancer and continue throughout the different stages of the cancer. Specifically, distinct tumour types often have characteristic patterns of methylation, which can be used as markers for early detection and/or monitoring the progression of carcinogenesis. For therapeutic purposes, the methylation of certain genes, particularly DNA repair genes, can cause sensitivity to specific chemotherapeutics and methylation of those genes can thereby act as a predictive marker if those chemotherapeutic agents are used.

A number of current methodologies for methylation studies already exist. For examples, sequencing of bisulphite-treated DNA is the gold standard for methylation studies as it reveals directly the status of each CpG dinucleotide. Other methodologies involves PCR amplification, such as in methylation specific PCR (MSP), where CpG specific oligonucleotide primers are used to distinguish between modified methylated and unmethylated nucleic acid. The identification of the methylated nucleic acid is based on the presence or absence of amplification product resulting from the amplification and distinguishing modified methylated and non-methylated nucleic acids.

Another methodology for determination of methylation status is methylation-sensitive melting curve analysis (MS-MCA) or high resolution melting curve analysis (HRMS-MCA). MS-MCA is a reliable technique, and the results do not need to be verified by other techniques, such as is required for example for positive MSP results. The MS-MCA technique is based on the fact that the melting temperature of methylated and unmethylated alleles are different after modification of unmethylated cytosine and amplification, which converts methylated C:G base pairs to A:T base pairs with a lower melting temperature. The standard protocol for determination of methylation status MS-MCA stipulates that the oligonucleotide primers used to amplify the target nucleic acid are devoid of CpG dinucleotides to ensure that the primers does not discriminate between methylated an unmethylated target alleles.

US 2009/0155791 A1 discloses an alternative method for determination of methylation status is methylation-sensitive melting curve analysis (MS-MCA) or high resolution melting curve analysis (HRMS-MCA). The employs an improved design of primers, methylation-independent oligonucleotide primers, that allows for the use of only one set of primers to detect both alleles of a CpG-containing nucleic acid after it has been subjected to C to T conversion by conventional techniques.

In order to determine the status of methylation of a cytosine of a CpG in a target sequence or the proportion of methylated target sequence of a polynucleotide in a biological sample, such application based method typically relies on the use of reference sequences, which are applied in control samples e.g. to set the baseline for an un-methylated state or a state of complete methylation.

The reference nucleic acid sequence in the form of polymerase amplified sequences is a frequent cause of cross-contamination, which required a thorough decontamination of the lab facility. There is therefore a need for improved reference nucleic acid sequence molecules for use in polymerase based methods for determining the status or proportions of methylated cytosine in a target sequences in a biological sample.

### SUMMARY OF THE INVENTION

The object of the present invention is to provide improved reference nucleic acid sequence for use in detecting a target nucleic acid, wherein the improved reference nucleic acid sequence reduces the risk of cross-contamination with reference nucleic acid sequence.

In a first aspect, the invention provides a set of vectors comprising
(i) a first vector comprising a vector backbone and a first reference nucleic acid sequence, wherein said first reference nucleic acid sequence comprises at least one CpG dinucleotide site, and wherein said reference nucleic acid sequence is having a sequence identical to or at least 95% identical to the corresponding length of a nucleic acid sequence selected from the group consisting a mammalian promoter, the 3′ downstream sequence of said promoter and 5′-upstream sequence of said promoter, and
(ii) a second vector comprising a vector backbone and a second reference nucleic acid sequence, wherein said a second reference nucleic acid sequence is a variant of the first reference nucleic acid sequence characterized the cytosine of said at least one CpG dinucleotide site of the first reference nucleic acid sequence have been substituted with a thymidine or a uracil nucleobase.

In a second aspect, the invention provides a kit comprising:

a set of vectors according to the invention, and

a set of oligonucleotide primers capable of hybridizing to said first and second reference nucleic acid sequence and suitable for amplification of said first and second reference nucleic acid sequence or a part thereof.

A further object of the present invention is an improved method for detecting the level of methylated cytosine in a target sequence of a polynucleotide, where the incidence of cross-contamination with reference nucleic acid sequence is reduced.

In a third aspect, the invention provides a method for detecting the methylation status of a cytosine of one or more in a target sequence of a polynucleotide comprising a mammalian promoter (preferably a human promoter), said method comprising the steps of
(a) providing a biological sample comprising a polynucleotide comprising a mammalian promoter containing a target sequence within said promoter, the 3′ downstream sequence of said promoter or 5′-upstream sequence of said promoter, wherein the target sequence comprises said one or more CpG dinucleotides,
(b) providing a first vector comprising a first reference nucleic acid sequence, wherein said reference nucleic acid sequence is identical to or at least 95% identical to said target sequence,
(c) providing second vector comprising a second reference nucleic acid sequence, wherein said second reference nucleic acid sequence is a variant of the first reference nucleic acid sequence characterized the cytosine of one or more CpG dinucleotides of the first reference nucleic acid sequence have been substituted with a thymidine or a uracil nucleobase,
(d) contacting said polynucleotide with an agent that converts cytosine nucleobases to uracil with proviso that any 5-methylcytosine nucleobases are unaffected by said agent,
(e) amplifying said target sequence using said at least one oligonucleotide primer and said polynucleotide as template,
(f) amplifying said first reference sequence using said at least one oligonucleotide primer and said first plasmid as template,
(g) amplifying said second reference sequence using said at least one oligonucleotide primer and said second plasmid as template,
(h) analysing and evaluating the methylation status of said one or more CpG dinucleotides of said polynucleotide using the product of the amplification of the first reference sequence as reference for a state of complete methylation and the product of the amplification of the second reference sequence as reference for a state of unmethylated or partly methylated of the target sequence.

Yet a further object of the present invention is an improved for detecting the proportion of methylated target sequence of a polynucleotide, where the incidence of cross-contamination with reference nucleic acid sequence is reduced.

In a fourth aspect, the invention provides a method for detecting the proportion of methylated target sequence of a polynucleotide comprising a mammalian promoter in a biological sample, said method comprising the steps of
(a) providing a biological sample comprising a polynucleotide comprising a mammalian promoter (preferably a human promoter) containing a target sequence within said promoter, the 3′ downstream sequence of said promoter or 5′-upstream sequence of said promoter, wherein said target sequence comprises at least one CpG dinucleotide,
(b) providing a first vector comprising a first reference nucleic acid sequence, wherein said reference nucleic acid sequence is identical to or at least 95% identical to said target sequence,
(c) providing second vector comprising a second reference nucleic acid sequence, wherein said a second reference nucleic acid sequence is a variant of the first reference nucleic acid sequence characterized the cytosine of all CpG dinucleotide site of the first reference nucleic acid sequence have been substituted with a thymidine or a uracil nucleobase,
(d) contacting said polynucleotide with an agent that converts cytosine nucleobases to uracil with proviso that any 5-methylcytosine nucleobases are unaffected by said agent,
(e) amplifying said target sequence using said at least one oligonucleotide primer and said polynucleotide as template,
(f) amplifying said first reference sequence using said at least one oligonucleotide primer and said first plasmid as template,
(g) amplifying said second reference sequence using said at least one oligonucleotide primer and said second plasmid as template,
(h) analysing and evaluating the proportion of methylated cytosine of said polynucleotide using the product of the amplification of the first reference sequence as reference for a state of complete methylation and the product of the amplification of the second reference sequence as reference for a state of a completely unmethylated target sequence.

### BRIEF DESCRIPTION OF THE DRAWINGS

FIG. 1 Schematic illustration of the principle behind HRM analysis. A) the difference in a DNA sequence after bisulfite conversion of a methylated and an unmethylated genomic region. B) the difference in melting properties of the PCR products from the methylated (right of the two curves) and the unmethylated (left of the two curves) templates.

FIG. 2 illustrates the target genomic sequence of MLH1 (untreated and bisulphite treated) and the primers applied in the assays of Example 1. Disclosed is further the MLH1 control sequences (methylated and unmethylated)

FIG. 3 Normalized melting curves illustrating methylation positive control, the assay calibration control, and methylation negative control of the gene specific templates supplied with the MethylDetect kit.

FIG. 4 Relative signal difference (d/dT) plot illustrating the methylation positive control, the assay calibration control, and methylation negative control of the gene specific templates.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

Vector

In the context of the present invention, the term vector refers to a DNA molecule used as a vehicle to artificially carry foreign genetic material into another cell, where it preferably can be replicated. Vectors includes plasmids, cosmids, phage vectors and viral vectors. The term vector backbone refers to the part of the vector that is configured to accept an insert nucleic acid sequence, such as the reference nucleic acid sequence disclosed herein. In one embodiment of the present invention, the vector backbone is selected from the group consisting of a plasmid, a cosmid, a phage vector, and a viral vector. In a preferred embodiment, the vector is a plasmid. The vector backbone of the vector preferably has origin of replication. The vector backbone of the vector preferably further has a selectable marker. Alternatively, the vector is prepared synthetically without replication in a host, with the proviso that the preparation does not involve any steps of amplification by primer extension.

The vector backbone serves a backbone for cloning of the reference nucleic acid sequence. The vector backbone preferably further has a multicloning site, wherein the reference nucleic acid sequence is inserted. Since it in the context of the present invention is not intended to express the cloned reference nucleic acid sequence, it is not required that the cloned reference sequence is operable linked to a promoter for expression e.g. in a bacterial host cell. Thus, in one embodiment, the reference nucleic acid sequence is not cloned in the vector backbone such that the sequence is operably linked to a promoter, such as a bacterial promoter. Thus in one embodiment, the vector is not an expression or transcription vector. In a preferred embodiment, the vector backbone is a plasmid comprising an origin of replication suitable for amplification of the plasmid in a bacterial host, such as E. coli. In another embodiment, the plasmid is selected from the group consisting of pIDTSmart (Amp) (SEQ ID NO: 8), pUCIDT (Amp) (SEQ ID NO: 7), pIDTSmart (Kan) (SEQ ID NO: 10), pUCIDT (Kan) (SEQ ID NO: 9), and pBRIDT (SEQ ID NO: 11).

The inventors have surprisingly discovered that the incidence of cross-contamination with reference nucleic acid sequence is markedly reduced when reference nucleic acid sequence provided with in the form of a vector of the invention comprising said reference sequence is applied as a reference. Previously applied reference nucleic acid sequence in the form of polymerase amplified sequences was a frequent cause of cross-contamination, which required a thorough decontamination of the lab facility. Without being bound by the theory, the inventors believe that the vectors of the present invention are less likely to be subject to spread by aerosols created during the process e.g. opening of tubes.

The vector backbone of the first vector and the second vector is preferably the same. In one embodiment, the vector backbone is having a size of at least 1000 bp, preferably at least 1500 bp, more preferably at least 1900 bp, such as in the range of 1900 bp to 3500 bp. In another embodiment, the vector backbone is plasmids, preferably having a size of at least 1900 bp, such as in the range of 1900 bp to 3500 bp, such as in the range of 2000 bp to 3000 bp.

CpG Dinucleotide

In the context of the present invention, the term “dinucleotide” refers to two sequential nucleotides. In particular, the dinucleotide CpG, which denotes a cytosine linked to a guanine by a phosphodiester bond, may be comprised in an oligonucleotide, and also comprised in a targeted sequence. CpG site is used herein as a reference to a CpG dinucleotide in a nucleic acid sequence.

Target Nucleic Acid Sequence

DNA methylation is a heritable, reversible and epigenetic change of the DNA sequence without altering its coding function. Methylation of DNA methylation potentially alters gene expression. Abnormal methylation of CpG islands associated with e.g. tumor suppressor genes may also cause decreased gene expression. Increased methylation of such regions may lead to progressive reduction of normal gene expression giving abnormal cells growth (i.e., a malignancy).

The CpG islands (CpG-rich sequences) are distributed across the human genome and often span the promoter region as well as the first exon of protein coding genes. Methylation of individual promoter region CpG islands usually turns off or reduce the rate of transcription by recruiting histone deacetylases, which supports the formation of inactive chromatin.

In the context of the present invention, a target sequence is a mammalian promoter or a CpG containing a target sequence within said promoter, the 3′ downstream sequence of said promoter or 5′-upstream sequence of said promoter or a fragment thereof. Thus in one embodiment, the target sequence is a mammalian promoter comprising at least one CpG. In another embodiment, the target sequence is a mammalian promoter comprising a CpG island. In a further embodiment, the target sequence is partial sequence of a mammalian promoter comprising at least one CpG, such as the CpG island or a partial sequence thereof. It is preferred that the target sequence is a human genomic sequence.

CpG islands are typically between 0.2 to about 1 kb in length and are located upstream of many housekeeping and tissue-specific genes, but may also extend into gene coding regions. Accordingly, target sequences also include sequences flanking the promoter and trans DNA elements, such as remotely located enhancer elements, which comprises at least one CpG site. In the context of the present invention, target sequences further include the 3′ downstream sequence of said promoter, where said target sequence comprises at least one CpG. An example of a 3′ downstream target sequence is the first exon of a protein coding mammalian gene. Target sequences may further include 5′-upstream sequence of said promoter. The target sequences may be the entire promoter sequence, the 3′ downstream sequence of said promoter or 5′-upstream sequence of said promoter. Typically, the target sequences is a partial sequences of the promoter sequence, the 3′ downstream sequence of said promoter or 5′-upstream sequence of said promoter, which comprises one or more CpG site, which are subject to the analysis using the methods of the present invention.

The target sequences comprises at least one CpG site. In another embodiment, the target sequences comprises at least two CpG dinucleotide sites. In a further embodiment, the target sequences comprises three, four, five, six, seven or eight CpG dinucleotide sites.

In one embodiment, the first reference sequence is a mammalian promoter comprising a CpG island. In a further embodiment, the target sequence is a partial sequences of a mammalian promoter sequence, the 3′ downstream sequence of said promoter or 5′-upstream sequence of said promoter, wherein the target sequence comprises at least 200 bp having a GC percentage greater than 50%, and an observed-to-expected CpG ratio greater than 60%, wherein the observed CpG is the number of CpG in the inserted sequence and the expected number CpGs is (G*C)/length of the inserted nucleic acid sequence.

The size of target sequences may vary according to the gene and the application. In one embodiment, target nucleic acid sequence is having a size in the range of 33 bp to 300 bp, such as 50 bp to 200 bp, such as 50 bp to 150 bp. In one embodiment, the size of the target nucleic acid sequence is in the range of 50 bp to 150 bp.

In a preferred embodiment, wherein promoter is a human promoter or the 3′ downstream sequence of a human promoter or 5′-upstream sequence of a human promoter.

Reference Nucleic Acid Sequence

The vector set of the present invention is suitable as references in methods for detecting the level of methylated cytosine in a target sequence of a polynucleotide comprising a mammalian promoter or for detecting the proportion of methylated target sequence of a polynucleotide comprising a mammalian promoter.

The reference nucleic acid sequences are inserted into the sequence of the vector backbone, preferably by cloning. Preferably, the vector comprises an origin of replication suitable for amplification in a suitable host organisms, e.g. a plasmid vector for amplification in a bacterial host, such as E. coli.

The first reference corresponds to the target sequence or comprises the target sequences. In a method that includes a step of contacting said polynucleotide with an agent (e.g. bisulphite) that converts cytosine nucleobases to uracil with proviso that any 5-methylcytosine nucleobases are unaffected by said agent, the first reference is a reference for complete methylation of all CpG cites in the target sequences. Thus, the first reference may be used as a reference of a state of complete methylation.

In one embodiment of the present invention, the first reference nucleic acid sequence comprises at least two CpG dinucleotide sites. In another embodiment, the first reference nucleic acid sequence comprises three, four, five, six, seven or eight CpG dinucleotide sites. In one embodiment, the first reference sequence is mammalian promoter comprising a CpG island or a partial sequence thereof.

In one embodiment, the first reference nucleic acid sequence is having a sequence identical to or at least 95% identical to the corresponding length of a nucleic acid sequence of a mammalian promoter, such as at least 97% identical to the corresponding length of a nucleic acid sequence of a mammalian promoter, for example at least 98% identical to the corresponding length of a nucleic acid sequence of a mammalian promoter, such as at least 99% identical to the corresponding length of a nucleic acid sequence of a mammalian promoter.

In another embodiment, the first reference nucleic acid sequence is having a sequence identical to or at least 95% identical to the corresponding length of a nucleic acid sequence of the 3′ downstream sequence of a mammalian promoter, such as at least 97% identical to the corresponding length of a nucleic acid sequence of the 3′ downstream sequence of a mammalian promoter, for example at least 98% identical to the corresponding length of a nucleic acid sequence of the 3′ downstream sequence of a mammalian promoter, such as at least 99% identical to the corresponding length of a nucleic acid sequence of the 3′ downstream sequence of a mammalian promoter.

In a further embodiment, the first reference nucleic acid sequence is having a sequence identical to or at least 95% identical to the corresponding length of a nucleic acid sequence of the 5′ upstream sequence of a mammalian promoter, such as at least 97% identical to the corresponding length of a nucleic acid sequence of the 5′ upstream sequence of a mammalian promoter, for example at least 98% identical to the corresponding length of a nucleic acid sequence of the 5′ upstream sequence of a mammalian promoter, such as at least 99% identical to the corresponding length of a nucleic acid sequence of the 5′ upstream sequence of a mammalian promoter.

In one embodiment, the first reference sequence is identical to or at least 95% identical to the corresponding length of a nucleic acid sequence of a mammalian promoter of said first plasmid comprises at least 200 bp having a GC percentage greater than 50%, and an observed-to-expected CpG ratio greater than 60%, wherein the observed CpG is the number of CpG in the inserted sequence and the expected number CpGs is (G*C)/length of the inserted nucleic acid sequence.

In a further embodiment, the first reference nucleic acid sequence is having a sequence identical to or at least 95% identical to the target sequence, such as at least 97% identical to target sequence, for example at least 98% identical to the target sequence, such as at least 99% identical to the target sequence.

The second reference nucleic acid sequence reference is used as reference for an un-methylated or partly un-methylated state of the target sequences. The second reference nucleic acid sequence is a variant of the first reference nucleic acid sequence characterized in that the cytosine of one or more CpG dinucleotide sites of the first reference nucleic acid sequence have been substituted with a thymidine (or uracil). Where second reference nucleic acid sequence is a variant of the first reference nucleic acid sequence characterized in that the cytosine of at least one but not all CpG dinucleotide sites (or a uracil nucleobase) have been substituted with a thymidine, such second reference nucleic acid sequence may be used as a reference for a state of partial methylation and/or CpG site specific methylation. Where second reference nucleic acid sequence is a variant of the first reference nucleic acid sequence characterized in that the cytosine of all CpG dinucleotide sites have been substituted with a thymidine (or a uracil nucleobase), such second reference nucleic acid sequence may be used as a reference for a state of a completely un-methylated target sequence.

Thus in one embodiment, the second reference nucleic acid sequence comprises a variant of the first reference nucleic acid sequence characterized the cytosine of all CpG dinucleotide sites of the first reference nucleic acid sequence have been substituted with a thymidine (or uracil).

In another embodiment, the second reference nucleic acid sequence comprises a variant of the first reference nucleic acid sequence characterized the cytosine of at least one but not all CpG dinucleotide sites of the first reference nucleic acid sequence have been substituted with a thymidine (or uracil).

In one embodiment, the first reference nucleic acid sequence comprises a CpG dinucleotide at or near the 5′ end of said reference sequence. In another embodiment, the first reference nucleic acid sequence comprises a CpG dinucleotide at or near the 3′ end of said reference sequence. In a further embodiment, the first reference nucleic acid sequence comprises two CpG dinucleotides at or near the 5′ end of said reference sequence. In yet a further embodiment, the first reference nucleic acid sequence comprises two CpG dinucleotides at or near the 3′ end of said reference sequence.

In one embodiment, the first reference nucleic acid sequence comprises a CpG dinucleotide positioned within the 5′ terminal 10 nucleotides of said reference sequence. In another embodiment, the first reference nucleic acid sequence comprises a CpG dinucleotide positioned within the 3′ terminal 10 nucleotides of said reference sequence. In yet another embodiment, the first reference nucleic acid sequence comprises a CpG dinucleotide positioned immediately 3′ to the 5′ terminal nucleotide of the oligonucleotide primer.

The size of the first (and second) reference nucleic acid sequence is typically about the same size as the target sequence. In one embodiment, the size of the reference nucleic acid sequence is same as the size of the target sequence. Thus, in one embodiment, the size of the first reference nucleic acid sequence is having a size in the range of 33 bp to 300 bp, such as 50 bp to 200 bp, such as 50 bp to 150 bp, which is the typical size of the target sequence. The size of the first (and second) reference nucleic acid sequence may be exceed the size of the target sequences, where the reference sequence includes sequences flanking the target sequence, which are not part of the target sequence.

An unlimited number of examples of promoters that may subject to analysis for CpG methylation are disclosed herein. In one embodiment, the promoter is a promoter of a gene selected from the group consisting of CHD 1 (cadherin 1, type 1, E-cadherin (epithelial)), COX2 (Cytochrome c oxidase subunit 2), PYCARD (PYD and CARD domain containing), BINI (Homo sapiens bridging integrator 1), BRCA1 (breast cancer 1), LATS2 (large tumor suppressor kinase 2), PITX2 (paired-like homeodomain 2), BCL2 (B-cell CLL/lymphoma 2), EYA4 (EYA transcriptional coactivator and phosphatase 4), GSK3B (glycogen synthase kinase 3 beta), MLH1 (EPM2A (laforin) interacting protein 1), TIMP-3 (synapsin III), MSH6 (mutS homolog 6), MTHFR (methylenetetrahydrofolate reductase (NAD(P)H)), PTEN (phosphatase and tensin homolog), SFN (stratifin), CD109 (CD109 molecule), ERS 1 (estrogen receptor 1), PCDH10 (protocadherin 10), DAPK1 (death-associated protein kinase 1), FHIT (fragile histidine triad), P1 6ink4a (Homo sapiens cyclin-dependent kinase inhibitor 2A), PRSS3 (protease, serine, 3), RASSF1 (Ras association (RaIGDS/AF-6) domain family), TMS 1 (Homo sapiens PYD and CARD domain containing), CAGE-1 (cancer antigen 1), GPR150 (G protein-coupled receptor 150), ITGA8 (integrin, alpha 8), PRDX2 (peroxiredoxin 2), SYK (spleen tyrosine kinase), ALX3 (ALX homeobox 3), HOXD11 (homeobox D11), PTPRO (protein tyrosine phosphatase, receptor type, O), WWOX (WW domain containing oxidoreductase), ABHD9 (epoxide hydrolase 3), CAV9 (Coxsackievirus A9), GPR78 (G protein-coupled receptor 78), GSTP1 (glutathione S-transferase pi 1), HIC1 (hypermethylated in cancer 1), PTGS2 (prostaglandin-endoperoxide synthase 2), CSMD1 (CUB and Sushi multiple domains 1), MGMT (O-6-methylguanine-DNA methyltransferase), BNIP3 (BCL2/adenovirus E1B 19 kDa interacting protein 3), PPP3CC CSMDI, MAP3k7 (mitogen-activated protein kinase kinase kinase 7), and C10orf59 (renalase, FAD-dependent amine oxidase).

In one embodiment, the promoter is a promoter of a gene selected from the group consisting of APC (Homo sapiens adenomatous polyposis coli (APC) NM_001127511), ATM (Homo sapiens ataxia telangiectasia mutated (ATM) NM_000051), MD_BRCA1 (Homo sapiens breast cancer 1, early onset (BRCA1) NM_007299), BRCA2 (Homo sapiens breast cancer 2, early onset (BRCA2) NM_000059), CA10 (Homo sapiens carbonic anhydrase X (CA10) NM_020178), CCND2 (Homo sapiens cyclin D2 (CCND2) NM_001759), CDH1 (Homo sapiens cadherin 1, type 1, E-cadherin (epithelial) (CDH1) NM_004360), CDH13 (Homo sapiens cadherin 13, H-cadherin (heart) (CDH13) NM_001220492), CDKN2B (Homo sapiens cyclin-dependent kinase inhibitor 2B (p15, inhibits CDK4) (CDKN2B) NM_004936), CTCF (Homo sapiens CCCTC-binding factor (zinc finger protein) (CTCF) NM_006565), DAPK1 (Homo sapiens death-associated protein kinase 1 (DAPK1) NM_004938), ESR1 (Homo sapiens estrogen receptor 1 (ESR1) NM_001122742), FHIT (Homo sapiens fragile histidine triad (FHIT) NM_002012), GHSR (Homo sapiens growth hormone secretagogue receptor (GHSR) NM_198407), GSTP1 Homo sapiens glutathione S-transferase pi 1 (GSTP1) NM_000852), H19 (Homo sapiens H19, imprinted maternally expressed transcript (non-protein coding) (H19) NR_002196), HIC1 (Homo sapiens hypermethylated in cancer 1 (HIC1) NM_006497), LHX1 (Homo sapiens LIM homeobox 1 (LHX1) NM_005568), LPL (Homo sapiens lipoprotein lipase (LPL) NM_000237), MGMT (Homo sapiens O-6-methylguanine-DNA methyltransferase (MGMT) NM_002412), MLH1 (Homo sapiens mutL homolog 1, colon cancer, nonpolyposis type 2 (E. coli) (MLH1) NM_000249), NR2E1 (Homo sapiens nuclear receptor subfamily 2, group E, member 1 (NR2E1) NM_003269), ONECUT2 (Homo sapiens one cut homeobox 2 (ONECUT2) NM_004852), P16 (Homo sapiens cyclin-dependent kinase inhibitor 2A (CDKN2A) NM_058197), PITX2 (Homo sapiens paired-like homeodomain 2 (PITX2), transcript variant 3 NM_000325), POU4F (Homo sapiens POU class 4 homeobox 2 (POU4F2) NM_004575), PTGER4 (Homo sapiens prostaglandin E receptor 4 (subtype EP4) (PTGER4) NM_000958), PTGS2 (Homo sapiens prostaglandin-endoperoxide synthase 2 (prostaglandin G/H synthase and cyclooxygenase) (PTGS2) NM_000963), RARB (Homo sapiens retinoic acid receptor, beta (RARB), transcript variant 1 NM_000965), RASSF1A (Homo sapiens Ras association (RaIGDS/AF-6) domain family member 1 (RASSF1) NM_170714), RUNX3 (Homo sapiens runt-related transcription factor 3 (RUNX3) NM_004350), Sept9 (Homo sapiens septin 9 (SEPT9) NM_001113493), SHOX2 (Homo sapiens short stature homeobox 2 (SHOX2) NM_003030), THBS1 (Homo sapiens thrombospondin 1 (THBS1) NM_003246), TIMP3 Homo sapiens TIMP metallopeptidase inhibitor 3 (TIMP3) NM_000362), TMS (Homo sapiens PYD and CARD domain containing (PYCARD) NM_013258), TP73 (Homo sapiens tumor protein p73 (TP73) NM_005427), and TWIST (Homo sapiens twist basic helix-loop-helix transcription factor 1 (TWIST1) NM_000474).

Oligonucleotide Primer(s)

The oligonucleotide primer used by the methods of the present invention and comprised in the kit of the present invention, are capable of hybridizing to both methylated and unmethylated nucleic acid alleles of the target sequence and modified as well as unmodified alleles (methylation-independent primer). The oligonucleotide used by the present invention is capable of being employed in amplification reactions, wherein the primers is used in amplification of a target sequence comprised in a template DNA originating from either a methylated or an unmethylated strand.

The preferred primer comprise a CpG dinucleotide. Accordingly, in a methylated and bisulfite modified nucleic acid target sequence, the primer sequence will anneal to the nucleic acid template with a perfect match, wherein all of the nucleotides in a consecutive region of the primer forms base pairs with a complementary region in the nucleic acid target. However, in an unmethylated nucleic acid target after bisulfite modification, the methylation-independent primers of the present invention will anneal to the nucleic acid template with an imperfect match, wherein the primer sequence comprise a mis-match (i.e. the primer and template does not form base pairs) at the position of the unmethylated Cytosine at a CpG site in the nucleic acid template. Nonetheless, as the primers used by the present invention are methylation-independent, the primers will hybridize to both unmethylated and methylated nucleic-acid sequences after bisulfite modification, and the primers will form a perfect match with the target sequence of a methylated nucleic acid target and an imperfect match, where the primers and target nucleic acid sequence does not form base pairing at the positions of unmethylated Cytosine (which is converted by bisulfite to Uracil) at CpG sites.

The oligonucleotide primer used by the methods of the present invention and comprised in the kit of the present invention will, due to the mis-match after bisulfite modification at positions of unmethylated cytosine of a CpG-site in the nucleic acid target sequence, hybridize less efficiently to an unmethylated nucleic acid sequence. However, by reducing the stringency of hybridization, primers used by the present invention are able to anneal to the nucleic acid target, also when the nucleic acid target comprise unmethylated CpG-sites, which have been modified by for example bisulfite treatment. In one example, the stringency is reduced by reducing the annealing temperature as described elsewhere herein.

The design of oligonucleotide primers suitable for nucleic acid amplification techniques, such as PCR, is known to people skilled within the art. The design of such primers involves analysis of the primer's melting temperatures and ability to form duplexes, hairpins or other secondary structures. Both the sequence and the length of the oligonucleotide primers are relevant in this context. The oligonucleotide primer may comprise between 10 and 100 consecutive nucleotides, such as 15 to 100, 15 to 90, 17 to 80, 18 to 70, 18 to 60. In one embodiment, oligonucleotide primer comprises between 15 and 60 consecutive nucleotides, such as 15 to 25 consecutive nucleotides, for example between 17 and 22 consecutive nucleotides, such as 18 to 22 consecutive nucleotides. In a specific embodiment, the oligonucleotide primers comprise between 17 and 22 consecutive nucleotides, such as 17, 18, 19, 20, preferably 21 or 22 consecutive nucleotides.

The oligonucleotide used by the methods of the present invention and included in the kit of the present invention typically has melting temperature in the ranges of 45 to 70 degrees Celsius, such as 50 to 65 degrees Celsius, such as 55 to 65 degrees Celsius.

Method of Determining CpG Methylation

In one aspect, the present invention provides a method for detecting the methylation status of one or more cytosine of a CpG dinucleotides in a target sequence of a polynucleotide comprising a mammalian promoter, said method comprising the steps of
(a) providing a biological sample comprising a polynucleotide comprising a mammalian promoter containing a target sequence within said promoter, the 3′ downstream sequence of said promoter or 5′-upstream sequence of said promoter, wherein the target sequence comprises said one or more CpG dinucleotides
(b) providing a first vector comprising a first reference nucleic acid sequence, wherein said reference nucleic acid sequence is identical to or at least 95% identical to said target sequence,
(c) providing second vector comprising a second reference nucleic acid sequence, wherein said second reference nucleic acid sequence is a variant of the first reference nucleic acid sequence characterized the cytosine of one or more CpG dinucleotides of the first reference nucleic acid sequence have been substituted with a thymidine or a uracil nucleobase,
(d) contacting said polynucleotide with an agent that converts cytosine nucleobases to uracil with proviso that any 5-methylcytosine nucleobases are unaffected by said agent,
(e) amplifying said target sequence using said at least one oligonucleotide primer and said polynucleotide as template,
(f) amplifying said first reference sequence using said at least one oligonucleotide primer and said first plasmid as template,
(g) amplifying said second reference sequence using said at least one oligonucleotide primer and said second plasmid as template,
(h) analysing and evaluating the methylation status of the cytosinse of said one or more CpG dinucleotides of said polynucleotide using the product of the amplification of the first reference sequence as reference for a state of complete methylation and the product of the amplification of the second reference sequence as reference for a state of partly methylation (or complete unmethylation) of said one or more CpG dinucleotides the target sequence.

In another aspect, the present invention provides, a method for detecting the proportion of methylated target sequence of a polynucleotide comprising a mammalian promoter in a biological sample, said method comprising the steps of
(a) providing a biological sample comprising a polynucleotide comprising a mammalian promoter containing a target sequence within said promoter, the 3′ downstream sequence of said promoter or 5′-upstream sequence of said promoter, wherein said target sequence comprises at least one CpG dinucleotide.
(b) providing a first vector comprising a first reference nucleic acid sequence, wherein said reference nucleic acid sequence is identical to or at least 95% identical to said target sequence,
(c) providing second vector comprising a second reference nucleic acid sequence, wherein said a second reference nucleic acid sequence is a variant of the first reference nucleic acid sequence characterized the cytosine of all CpG dinucleotide site of the first reference nucleic acid sequence have been substituted with a thymidine or a uracil nucleobase,
(d) contacting said polynucleotide with an agent that converts cytosine nucleobases to uracil with proviso that any 5-methylcytosine nucleobases are unaffected by said agent,
(e) amplifying said target sequence using said at least one oligonucleotide primer and said polynucleotide as template,
(f) amplifying said first reference sequence using said at least one oligonucleotide primer and said first plasmid as template,
(g) amplifying said second reference sequence using said at least one oligonucleotide primer and said second plasmid as template,
(h) analysing and evaluating the proportion of methylated cytosine of said polynucleotide using the product of the amplification of the first reference sequence as reference for a state of complete methylation and the product of the amplification of the second reference sequence as reference for a state of a completely unmethylated target sequence.

Biological Sample

The biological sample provided for analysis by the methods of the present invention may be a biological obtained from a biological sample of any source. In a preferred embodiment, the sample is obtained from a human subject. In one embodiment, the biological sample is selected from the group consisting of solid tissue, blood, serum and body fluids. In another embodiment, the biological sample is selected from the group consisting of breast tissue, ovarian tissue, uterine tissue, bladder tissue, colon tissue, prostate tissue, lung tissue, renal tissue, thymus tissue, testis tissue, hematopoietic tissue, bone marrow, urogenital tissue, expiration air, stem cells (such as cancer stem cells), sputum, urine, blood and sweat.

Agents Modifying Unmethylated Cytosine

The method of the present invention preferably uses an agent, which modifies unmethylated cytosine in the CpG-containing nucleic acid. The methods of the present invention includes a process step of contacting the polynucleotide comprised in the biological sample with an agent that converts any unmethylated cytosine nucleobase to another nucleobase, which will distinguish an unmethylated cytosine from a methylated cytosine. In the process step any 5-methylcytosine nucleobases are unaffected by said agent.

In one preferred embodiment, an agent modifies unmethylated cytosine to uracil. Such an agent may be any agent conferring said conversion, wherein unmethylated cytosine is modified, but not methylated cytosine. In one preferred contacting said polynucleotide with an agent that converts cytosine nucleobases to uracil with proviso that any 5-methylcytosine nucleobases are unaffected by said agent. In one embodiment, the agent for modifying unmethylated cytosine is bisulphite, such as sodium bisulphite. Sodium bisulphite (NaHSO₃) reacts readily with the 5,6-double bond of cytosine, but only poorly with methylated cytosine. The cytosine reacts with the bisulfite ion forming a reaction intermediate in the form of a sulfonated cytosine which is prone to deamination, eventually resulting in a sulfonated uracil. Uracil can subsequently be formed under alkaline conditions, which removes the sulfonate group. In a preferred embodiment, the agent (converting unmethylated cytosine) is bisulphite, such as sodium bisulphite.

Target Amplification Step

The methods of the present invention comprises a process step of amplifying the target sequence comprised in the polynucleotide of the biological samples, which have been subjected to an agent, which modifies unmethylated cytosine in the CpG-containing nucleic acid. Preferably simultaneously, but in a separate reaction(s) the amplification of the reference sequence of the corresponding first and second reference sequence under the same reactions conditions and using the same one oligonucleotide primer or set of one oligonucleotide primers. Thus in one embodiment, the target and reference sequences are amplified using a set of primers capable of hybridizing to said templates and reference sequences and amplify said target and reference sequences. The template is preferable a DNA polynucleotide. Although it is preferred that the amplification of the target sequence of the biological sample and the reference sequences is performed simultaneously, it may be performed independently, e.g. the amplification of the target sequence of the reference samples may be performed separately and analysed separately. The data obtained may be used as reference in the methods described herein as had they been run simultaneously.

In one embodiment, the target sequence and reference sequences are amplified by a primer extension reaction. Preferably, the amplification is done by a PCR reaction. Thus, in one embodiment, the primer extension reaction is PCR. During a nucleic acid amplification process uracil will by the Taq polymerase be recognised as a thymidine. The product upon PCR amplification of a sodium bisulfite modified nucleic acid contains cytosine at the position where a methylated cytosine (5-methylcytosine) occurred in the starting template DNA of the sample. Moreover, the product upon PCR amplification of a sodium bisulfite modified nucleic acid contains thymidine at the position where an unmethylated cytosine (5-methylcytosine) occurred in the starting template DNA of the sample. Thus, an unmethylated cytosine in converted into a thymidine residue upon amplification of a bisulfite modified nucleic acid.

The amplification of the target sequence typically includes three step: (i) a denaturation step, where the strands of the template is separated (melted) under high temperature conditions; (ii) an annealing step, where the oligonucleotides primer(s) are allowed to hybridize to the template by forming hydrogen bonds with the template. Typical heat denaturation involves temperatures ranging from about 85 degrees Celsius to 102 degrees Celsius for times ranging from about 1 to 10 minutes.

Annealing Temperature

After the denaturation step, the oligonucleotides primer(s) are allowed to hybridize to the template. The annealing is facilitated by adjusting the temperature of the reaction to be about the melting temperature of the primers.

Other factors than annealing temperature also affect hybridisation to a CpG-containing target sequence of a methylation-independent primer according to the present invention. At highly stringent conditions, hybridization between perfect matching primer and target sequences are favoured, such as hybridization between a methylation-independent primer according to the present invention and a methylated target sequence upon cytosine modification. Less stringent conditions will tend to favour oligonucleotide primer binding, priming and amplification of the unmethylated allele. Modulation of temperature is one way of adjusting the stringency of hybridization, but the stringency of hybridization may also be modulated by adjusting buffer composition, and/or salt concentrations in the hybridization mixture, which is known to those of skill within the art. The present invention comprises any such method of modulating hybridization stringency to balance the PCR bias towards amplification of unmethylated template. However, modulation of temperature is preferred.

In one embodiment of the present invention, the primer annealing temperature during amplification of said target sequence and reference sequences is in the range of 40 and 75 degrees Celsius, such as in the range of 45 to 70 degrees Celsius, for examples in the range of 50 to 65 degrees Celsius, such as in the range of 55 to 65 degrees Celsius. In a specific embodiment, the annealing temperature is 60 degrees Celsius or about 60 degrees Celsius. In another specific embodiment, the annealing temperature is 64 degrees Celsius or about 64 degrees Celsius.

Enzymes that are suitable for amplification include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (such as Taq polymerases). Suitable enzymes will facilitate combination of the nucleotides in the proper manner to form the primer extension products, which are complementary to each locus nucleic acid strand. Generally, the synthesis will be initiated at the 3′ end of each primer and proceed in the 5′ direction along the template strand, until synthesis terminates generating molecules of different lengths. There may be agents for polymerization, however, which initiate synthesis at the 5′ end and proceed in the other direction, using the same process as described above.

The oligonucleotide primers annealed to the template is elongated to form an amplification product. The elongating temperature depends on optimum temperature for the polymerase, and is usually between 30 and 80 degrees Celsius. Typically, the elongating temperature is between 60 and 80 degrees Celsius. The elongation time depends on the size of the target sequence. Typically, the PRC reaction mixture is incubated at the elongating temperature for 1 to 100 seconds, such as 10 to 100 seconds, such as 20 to 100 seconds, such as 30 to 100 seconds, such as 40 to 100 seconds or such as 50 to 100 seconds.

The amplification reaction is performed in a buffered aqueous solution, preferably at a pH of 7-9. The oligonucleotide primer(s) are added to the reaction mixture in a molar excess of primer: template especially when the template is genomic DNA which will ensure an improved efficiency. Deoxyribonucleoside triphosphates dATP, dCTP, dGTP, and dTTP are added to the reaction mixture, either separately or together with the primers.

The amplification of the target sequence comprises sequentially denaturation of the template, annealing of the oligonucleotide primer(s), and elongation of the primer(s). This sequence is done for a number of cycles, typically between 10 and 70 cycles, such as between 25 and 55 cycles.

### REFERENCES

The first and second vectors may be applied and subjected to amplification of the reference sequence in separate amplification reference reactions. Typically, the reference reaction comprises a mixture of the first and the second vector of the present invention. Thus in one embodiment, step (f) and (g) is performed in a reaction comprising said mixture of said first vector and said second vector. Thus, in one embodiment, said first and said second vector is provided in the form of a mixture of said first and said second vector. In a preferred embodiment, said mixture is obtained by preparing a serial dilution of said first vector into said second vector.

For the method for detecting the proportion of methylated target sequence of a polynucleotide comprising a mammalian promoter in a biological sample, a serial dilution of the first reference in the second reference is particular useful. By diluting the first reference sequence (corresponding to a unmethylated allele) into a background of the second reference sequence (corresponding to a methylated allele), each of the dilutions may be used a reference for a particular proportion of methylated target sequence. Although a single of such dilutions may be employed in the method, a plurality of reference samples obtained by a serial dilution is preferably used. The plurality of reference samples facilitates the detecting the proportion of methylated target sequence. For example, the proportion (relative amount) of methylated CpG sites may evaluated by comparison with melting curve analysis of the product of the amplification a plurality of reference samples obtained by the serial dilution of said first vectors into said second vectors. Thus in one embodiment, step (f) and (g) is performed on a plurality of reference samples obtained by a serial dilution of said first vector into said second vector. In a preferred embodiment, the amount of template used in (e), (f) and (g) is the same or essentially the same. For example, if the amplification of the reference sequences is performed on a reference sample, which is a mixture of the first vector and said second vector, the total amount of template in the reference sample is the same or approximately the same as the total amount of template in the biological sample.

The serial dilution is typically prepared by diluting the first vector (comprising the reference sequence corresponding to an unmethylated allele) into a background of the second vector (comprising the reference sequence corresponding to a methylated allele). It may also be done the other way around, i.e. diluting the second vector into the first vector.

In one embodiment, the plurality of reference samples obtained by a serial dilution of said first vector into said second vector comprises reference samples comprising 0% to 100% of said first vector. In another embodiment, the plurality of reference samples obtained by a two-fold serial dilution of said first vector into said second vector. In a further embodiment, the plurality of reference samples obtained by a five-fold serial dilution of said first vector into said second vector. In yet a further embodiment, the plurality of reference samples obtained by a ten-fold serial dilution of said first vector into said second vector.

Analysis of Product of Amplification

The product obtained by the amplification of the target sequence in the polynucleotide of the biological sample and the first and second reference vectors is subsequently subjected to analysis and evaluation step. The difference in nucleic acid sequence at previously methylated or unmethylated cytosines allows for the analysis of methylation status in a sample.

In one embodiment, the analysis and evaluation is performed using a method selected from the group consisting of melting curve analysis, high-resolution melting analysis, nucleic acid sequencing, primer extension, denaturing gradient gel electrophoresis, southern blotting, restriction enzyme digestion, methylation-sensitive single-strand conformation analysis (MS-SSCA) and denaturing high performance liquid chromatography (DHPLC).

In a preferred embodiment, the analysis and evaluation step (h) is performed using melting curve analysis. In another embodiment, the analysis of the amplified target sequence is performed by high resolution melting analysis (HRM). Analysis and evaluation of methylated and unmethylated alleles by melting curve analysis is disclosed US 2009/0155791, the method of which is incorporated by reference in this application.

Melting curve analysis or high resolution melting analysis exploits the fact that methylated and unmethylated alleles are predicted to differ in thermal stability because of the difference in GC contents after bisulphite treatment and PCR-mediated conversion of methylated C:G base pairs to A:T base pairs. The melting temperature of an amplification product according to the present invention is determined by the composition of methylated and unmethylated alleles in the nucleic acid sample. If the nucleic acid is completely unmethylated, all cytosines are converted to thymines, and the resulting PCR product will have a relatively low melting temperature compared to a methylated nucleic acid. If on the other hand, the nucleic acids comprised in the sample contain methylated cytosines at all the CpG dinucleotides, the melting temperature of the PCR product will be relatively higher. If the nucleic acid sample comprises a mixture of methylated and unmethylated alleles, bisulphite treatment followed by amplification will result in two distinct amplification products. The unmethylated alleles will display a low melting temperature and the methylated alleles a high melting temperature.

If only a subset of the CpG dinucleotides of the target sequence contain a methylated cytosine, the amplification product represents a pool of molecules with different melting temperatures, which leads to an overall intermediate melting temperature.

Melting curve analysis is performed by incubating the nucleic acid amplification product at a range of increasing temperatures. The temperature is increased from a starting e.g. temperature of at least 50 degrees Celsius, such as 60 or 70 degrees Celsius. The temperature is then increased to a final temperature of e.g. at least 70 degrees Celsius, such as 80 to 100 degrees Celsius. In one embodiment, the melting curve analysis is performed by incubating the nucleic acid amplification product at increasing temperatures, from 70 to 95 degrees Celsius, wherein the temperature increases by 0.05 degrees per second. In one embodiment, the melting curve analysis is performed by using a thermal cycler.

The melting of the nucleic acid can be measured by a number of methods, which are known to people within skill of the art. One method involves use of agents, which fluoresce when bound to a nucleic acid in its double stranded conformation. Such agents include fluorescent probes or dyes, such as ethidium bromide, EvaGreen, LC Green, Syto9, SYBR Green, SensiMix HRMTm kit dye. Thus, in one embodiment, the melting curve analysis is performed by measurement of fluorescence. The melting of the nucleic acid amplification product can then be monitored as a decrease in the level of fluorescence from the sample. After measurement of the fluorescence the melting curves can be generated by plotting fluorescence as a function of temperature. In one embodiment, the melting curve analysis is performed by using a thermal cycler in combination with a fluorometre.

For direct comparison of melting curves from samples that have different starting fluorescence levels, the melting curves for data collected in HRM can be normalized, as described in the examples of the present invention. Such normalization methods are known to people of skill in the art. One preferred means of normalization include calculation of the ‘line of best fit’ in between two normalization regions before and after the major fluorescence decrease representing the melting of the amplification product. The ‘line of best fit’ is a statistical measure, designating a line plotted on a scatter plot of data (using a least-squares method) which is closest to most points of the plot. In one embodiment, the melting curve analysis comprise normalization of melting curves by calculation of the ‘line of best fit’ in between two normalization regions before and after a major fluorescence decrease.

The melting curve analysis allows the determination of the relative amount of methylated CpG-containing nucleic acid in a sample. By comparison of the melting curve of an product of the biological sample used by the method of the invention with the melting curve of at least one reference sample comprising a mixture of the first and second vector, the relative amount of methylated CpG-containing nucleic acid can be estimated.

Thus in one embodiment, the relative amount (proportion) of methylated CpG sites is evaluated by comparison with melting curve analysis of the product of the amplification of at least one first reference, said second reference or a mixture of said first reference and second reference. In another embodiment, the relative amount (proportion) of methylated CpG sites is evaluated by comparison with melting curve analysis of the product of the amplification a plurality of reference samples obtained by a serial dilution of said first vector into said second vector.

Where the melting temperature (as measured by the melting curve) of an biological sample is higher than the melting temperature of a reference sample comprising the product of amplification of the first and second vector, then the relative amount of methylated CpG-containing nucleic acid in said biological sample is also higher than the relative amount of methylated CpG-containing nucleic acid in the reference sample. Conversely, if the melting curve of a biological sample is lower, i.e. the melting temperature is lower, than the melting temperature of a reference sample, then the relative amount of methylated CpG-containing nucleic acid in said biological sample is also lower than the relative amount of methylated CpG-containing nucleic acid in the reference sample. The amount of reference samples included in the melting curve analysis, thus determines the precision of the determination of methylation status. The more reference samples, the more precise can the relative amount of nucleic acids be determined.

Thus, in one embodiment of the present invention a higher melting temperature of the amplified nucleic acid of the biological sample than of the reference sample is indicative of a higher relative amount of methylated nucleic acid of that sample than of the reference sample. Conversely, a lower melting temperature of the amplified nucleic acid biological sample than of the reference is indicative of a lower relative amount of methylated nucleic acid of that sample than of the reference sample.

The term “peak melting temperature” as used herein, refers to the temperature at which the largest discrete melting step occurs. The nature of nucleic acid melting is explained elsewhere herein. A nucleic acid sample subjected to melting curve analysis may display more than one peak melting temperature. In a preferred embodiment of the present invention, the melting curve analysis display at least 1, 2 or 3 peak melting temperatures. In another embodiment, a melting profile displays at least one peak melting temperature. In a further embodiment, a melting profile displays at least two peak melting temperatures. In yet a further embodiment, the peak melting temperature corresponds to the highest level of the negative derivative of fluorescence (−dF/dT) over temperature versus temperature (T).

Kit of Parts

In a further aspect, the present invention provides a kit of parts comprising a set of vectors of the present invention (a first and a second vector) and a set of oligonucleotide primers capable of hybridizing to said first and second reference nucleic acid sequence and, wherein said set of oligonucleotide primers are suitable for amplification of said first and second reference nucleic acid sequence or a partial sequence thereof.

In one embodiment, the at least one of the oligonucleotide primers comprises at least one CpG dinucleotide, such as at least two CpG dinucleotides. In another embodiment, one of the oligonucleotide primers comprises a CpG dinucleotide at or near the 5′ end of the oligonucleotide primer. In a further embodiment, one of the oligonucleotide primers comprises a CpG dinucleotide at or near the 5′ end of the oligonucleotide primer. In yet another embodiment, one of the oligonucleotide primers comprises two CpG dinucleotides at or near the 5′ end of the oligonucleotide primer. In one embodiment, one of the oligonucleotide primers comprises two CpG dinucleotides at or near the 5′ end of the oligonucleotide primer. In another embodiment, one of the oligonucleotide primers comprises a CpG dinucleotide positioned within 5′ terminal 10 nucleotides of the oligonucleotide primer. In a further embodiment, one of the oligonucleotide primers comprises a CpG dinucleotide positioned within 5′ terminal 10 nucleotides of the oligonucleotide primer. In yet a further embodiment, one of the oligonucleotide primers comprises a CpG dinucleotide positioned immediately 3′ to the 5′ terminal nucleotide of the oligonucleotide primer. In one embodiment, one of the oligonucleotide primers comprises a CpG dinucleotide positioned immediately 3′ to the 5′ terminal nucleotide of the oligonucleotide primer.

The kit comprises at least one set of oligonucleotide primers capable of hybridizing to said first and second reference nucleic acid sequence and suitable for amplification of said first and second reference nucleic acid sequence or a part thereof. The size of the oligonucleotides typically depends on the target sequence and the application. In one embodiment, the oligonucleotide primers are having a size in the range of 10 to 100 nucleotides, such as 15 to 60 nt, such as 15 to 25 nt, such as 17 to 22 nt, such as 18 to 22 nt. The oligonucleotides primers of the kit typically have the same size or about the same size.

In one embodiment, the oligonucleotide primers are having melting temperature in the ranges of 45 to 70 degrees Celsius, such as 50 to 65 degrees Celsius, such as 55 to 65 degrees Celsius.

The kit may include one or more reagents for use in carrying out the methods of the present invention.

The method of the present invention uses an agent, which is capable of modifying unmethylated cytosine in the CpG-containing nucleic acid. The kit of the present invention may therefore include an agent that is capable of modifying unmethylated cytosine nucleobases e.g. to uracil. In one embodiment, the agent for modifying unmethylated cytosine is bisulphite, such as sodium bisulphite.

Although agents that modifies unmethylated cytosine in the CpG-containing nucleic acid, the kit may comprise an agent that is capable of modifying methylated cytosine nucleobases for applications, which rely on detecting modified methylated cytosine nucleobases.

The kit may include further reagents. In one embodiment, the kit further comprises at least one reagent selected from the group consisting of a deoxyribonucleoside triphosphate, a DNA polymerase enzyme and reaction buffer suitable for nucleic acid amplification.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended by the inventors to be optionally substitutable with the terms “consisting of”, “consist of” and “consists of”, respectively, in every instance.

### EXAMPLES

### Example 1—MLH1

The present example was performed on the LightCycler® 480 High Resolution Melting platform (product number: 05015278001) and LightCycler® 480 High Resolution Melting Master (product number: 04909631001) PCR reagents in 96 well plates. Although this platform is preferred other platforms may be used.

Assay Time

The cycling program includes 10 min pre-incubation and 50 cycles of amplification followed by high resolution melting and will last for approximately 90-120 min when performed using the LightCycler® 480 System.

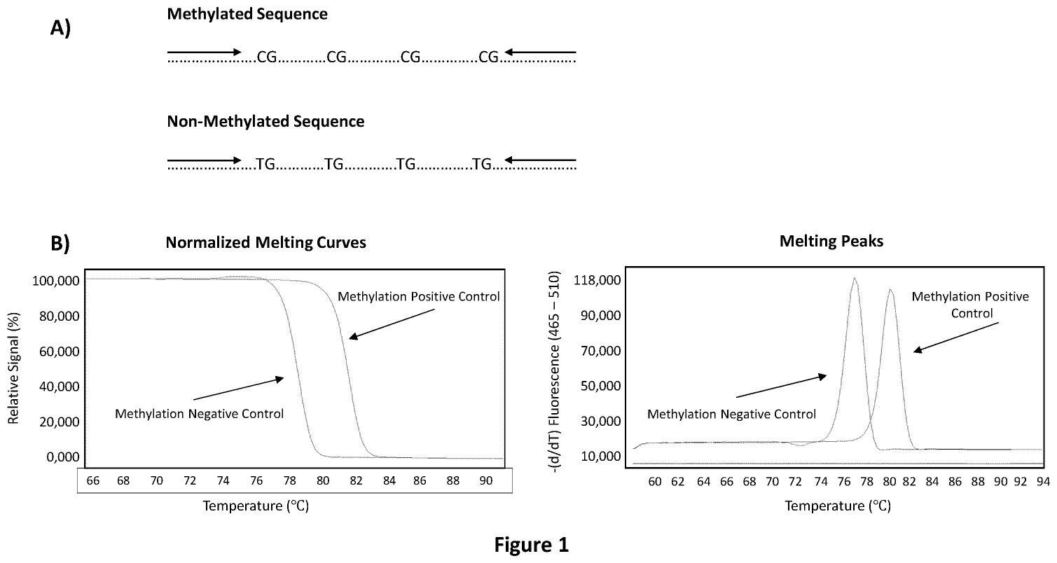

All DNA samples were subjected to bisulfite treatment, which converts unmethylated cytosines to uracils converted while preserving methylated cytosines in the template. The unmethylated cytosines, converted to uracil, is substituted with thymine during the PCR. After bisulfite conversion the DNA strands are no longer complementary, and will appear single-stranded. After PCR, the products will have different melting properties, and the resulting profile after HRM allow for discrimination between methylated and unmethylated templates, respectively (FIG. 1).

For each reaction 50-100 ng of bisulfite modified DNA is used. This is a theoretical concentration based on the DNA input for bisulfite conversion and the elution volume. Commercially kits are available for bisulfite conversion of the sample DNA (e.g. the bisulfite conversion kits from Zymo Research).

The quality of the DNA should be suitable for PCR in terms of concentration, purity and absence of PCR inhibitors. Use of same DNA extraction procedure for all samples may eliminate any subtle differences in the high-resolution melting results, which could have been introduced by a difference in reagent components. To ensure sufficient quality of the DNA prior to bisulfite conversion, agarose gel electrophoresis or analysis by a Bioanalyzer can be used to assess the DNA integrity, and Qubit Fluorometer is recommended for measuring the DNA concentration.

Assay Calibration Controls

A methylation positive a methylation negative control was used for an assay calibration to ensure the assay sensitivity to detect methylation of 1%. The controls were applied in duplicates or triplicates to all multi-well plates.

Negative Control Reaction

A No Template Control (NTC) was included in the analysis. The NTC contained the same reagents as the reactions for analyses, except that the DNA sample was replaced with the same amount of PCR grade water. The NTC was present at each multi-well plate in duplicates or in triplicates.

Primers

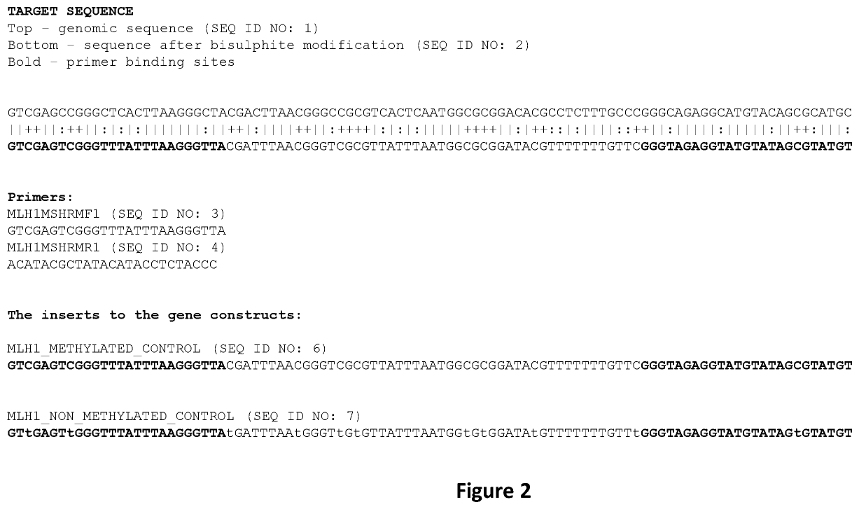

The primers used for the assay are disclosed on FIG. 2.

The protocol was calibrated using the LightCycler® 480 High Resolution Melting Master. 20 μl standard reaction was prepared according to the protocol below.

1. Thaw the solutions and spin all tubes briefly in a micro-centrifuge before opening, to ensure that the content is collected at the bottom of the tube. —store all reagents on ice.

2. Prepare the PCR mix for one 20 μl reaction by adding the following components in the order listed below and keep it on ice.

TABLE 1

| Component | Volume |
|---|---|
| HRM Master 2 × conc. | 10 μl |
| Primer mix | 1.0 μl |
| MgCl2 (25 mM) | 2.4 μl |
| H₂O (PCR grade) | 0.6 μl |
| In total | 14 μl |

3. Mix the reagents carefully by pipetting up and down and spin briefly. Do not vortex

4. Pipette 14 μl PCR mix into each well, including the wells to contain the positive, the assay calibration, the negative, and the no template controls.

5. Add 6 μl bisulfite treated DNA, corresponding to a theoretical calculated value of 50-100 ng DNA. For optimal performance, the amount of template was tested in the range of 50-100 ng. Lower amount of template can be used however, it is recommended that the assay is optimized to the specific DNA concentration before processing the test samples. For each multi-well plate, add 6 μl of each standard control DNA (methylation positive, assay calibration, and methylation negative), preferably in triplicates.

6. Seal the multiwell plate with an appropriate sealing foil.

7. Spin for 2 min at 1000×g.

8. Place the multiwell plate in the instrument and start the PCR-HRM program.

TABLE 2

| | | | | Ramp | |
|---|---|---|---|---|---|
| | | Temperature | Hold | Rate | Acquisitions |
| Program | Cycles | (° C.) | (sec) | (° C./sec) | (per ° C.) |
| Pre- | 1 | 95 | 600 | | |
| Incubation | | | | | |
| Amplification | 50 | 95 | 15 | 4.4 | None |
| | | 59-61 | 10 | 2.2 | None |
| | | 72 | 15 | 4.4 | Single |
| High | 95 | 15 | 4.4 | None | |
| Resolution | 60 | 60 | 2.2 | None | |
| Melting | 95 | Continuous | 0.01 | 50 | |

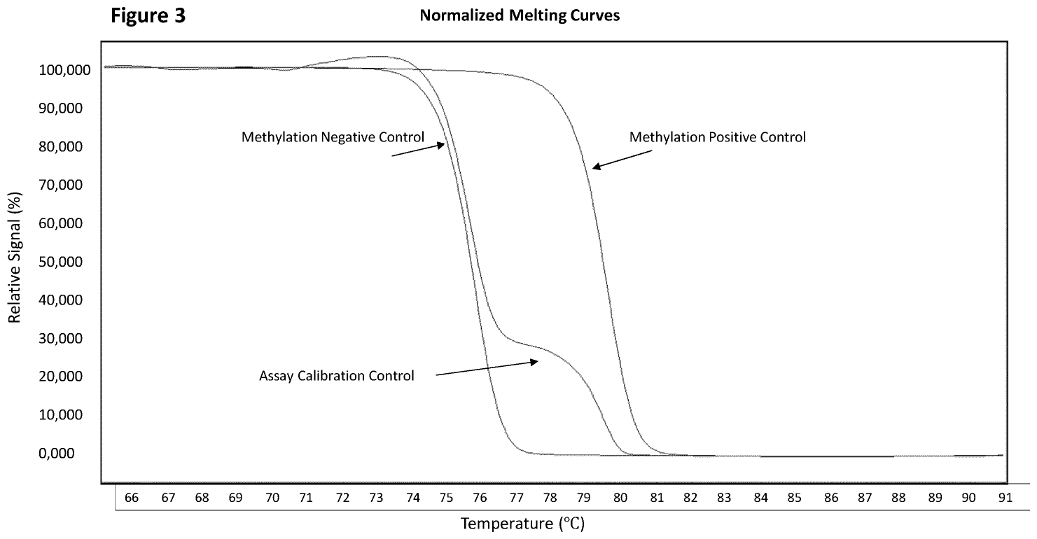

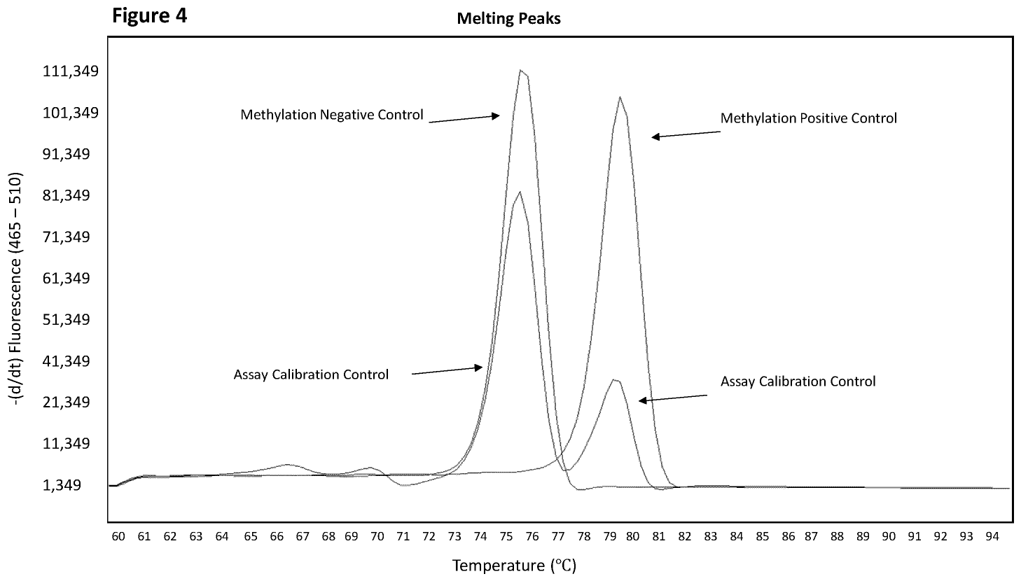

The results presented in FIGS. 3 and 4 were obtained following above protocol, using the LightCycler® 480 High Resolution Melting Master. After the amplification part of the program, the amplicons are analysed by high resolution melting curve analysis, and the data evaluated using the LightCycler® Gene scanning software.

Methylation-Sensitive High-Resolution Melting (MS-HRM) is a high-throughput technology for highly sensitive DNA methylation analysis of single loci. The technology utilizes the difference in melting properties of the PCR product amplified from methylated and unmethylated DNA strands after bisulfite conversion. The inclusion of standard DNA with known DNA methylation status ensures a highly sensitive read-out of the methylation of the test DNA. MS-HRM was shown to differentiate between methylated, un-methylated, and heterogeneous methylated templates, which have clearly distinguishable profiles after High-Resolution Melting (HRM).

1. A set of vectors comprising:
(i) a first vector comprising a vector backbone and a first reference nucleic acid sequence, wherein said first reference nucleic acid sequence comprises at least one CpG dinucleotide site, and wherein said first reference nucleic acid sequence comprises a sequence identical to or at least 95% identical to the corresponding length of a nucleic acid sequence selected from the group consisting a mammalian promoter, the 3′ downstream sequence of said promoter and 5′-upstream sequence of said promoter, and
(ii) a second vector comprising a vector backbone and a second reference nucleic acid sequence, wherein said a second reference nucleic acid sequence is a variant of the first reference nucleic acid sequence, wherein the cytosine of said at least one CpG dinucleotide site of the first reference nucleic acid sequence has been substituted with a thymidine or a uracil nucleobase.

2-70. (canceled)

71. The set of vectors according to claim 1, wherein the vector backbone of said first or second vector is selected from the group consisting of a plasmid, a cosmid, a phage vector, and a viral vector.

72. The set of vectors according to claim 1, wherein the vector backbone of said first or second vector has a size of at least 1000 bp.

73. The set of vectors according to claim 1, wherein said vector backbone of said first or second vector is a plasmid, having a size of at least 1900 bp.

74. The set of vectors according to claim 1, wherein vector backbone of said first or second vector is a plasmid selected from the group consisting of pIDTSmart (Amp) (SEQ ID NO: 8), pUCIDT (Amp) (SEQ ID NO: 7), pIDTSmart (Kan) (SEQ ID NO: 10), pUCIDT (Kan) (SEQ ID NO: 9), and pBRIDT (SEQ ID NO: 11).

75. The set of vectors according to claim 1, wherein said first reference nucleic acid sequence comprises at least two CpG dinucleotide sites.

76. The set of vectors according to claim 1, wherein said first reference nucleic acid sequence comprises three, four, five, six, seven or eight CpG dinucleotide sites.

77. The set of vectors according to claim 1, wherein said first reference nucleic acid sequence has a sequence identical to or at least 95% identical to the corresponding length of a nucleic acid sequence of a mammalian promoter.

78. The set of vectors according to claim 1, wherein said first reference nucleic acid sequence has a sequence identical to or at least 95% identical to the corresponding length of a nucleic acid sequence of the 3′ downstream sequence of a mammalian promoter.

79. The set of vectors according to claim 1, wherein said first reference nucleic acid sequence has a sequence identical to or at least 95% identical to the corresponding length of a nucleic acid sequence of the 5′ upstream sequence of a mammalian promoter.

80. The set of vectors according to claim 1, wherein said mammalian promoter comprises a CpG island.

81. The set of vectors according to claim 1, wherein said first reference sequence is identical to or at least 95% identical to the corresponding length of a nucleic acid sequence of a mammalian promoter of said first plasmid comprises at least 200 bp having a GC percentage greater than 50%, and an observed-to-expected CpG ratio greater than 60%, wherein the observed CpG is the number of CpG in the inserted sequence and the expected number CpGs is (G*C)/length of the inserted nucleic acid sequence.

82. The set of vectors according to claim 1, wherein the second reference nucleic acid sequence comprising a variant of the first reference nucleic acid sequence, wherein the cytosine of all CpG dinucleotide sites of the first reference nucleic acid sequence have been substituted with a thymidine or a uracil nucleobase.

83. The set of vectors according to claim 1, wherein first reference nucleic acid sequence comprises a CpG dinucleotide at or near the 5′ end of said reference sequence.

84. The set of vectors according to claim 1, wherein first reference nucleic acid sequence comprises a CpG dinucleotide at or near the 3′ end of said reference sequence.

85. The set of vectors according to claim 1, wherein first reference nucleic acid sequence comprises two CpG dinucleotides at or near the 5′ end of said reference sequence.

86. The set of vectors according to claim 1, wherein first reference nucleic acid sequence comprises two CpG dinucleotides at or near the 3′ end of said reference sequence.

87. The set of vectors according to claim 1, wherein first reference nucleic acid sequence comprises a CpG dinucleotide positioned within the 5′ terminal 10 nucleotides of said reference sequence.

88. The set of vectors according to claim 1, wherein first reference nucleic acid sequence comprises a CpG dinucleotide positioned within the 3′ terminal 10 nucleotides of said reference sequence.

89. The set of vectors according to claim 1, wherein the first reference nucleic acid sequence comprises a CpG dinucleotide positioned immediately 3′ to the 5′ terminal nucleotide of the oligonucleotide primer.
